(19) United States
(12) Patent Application Publication
(10) Pub. No.: US 2011/0201936 A1
(43) Pub. Date: Aug. 18, 2011

(54) ULTRASOUND PROBE

(75) Inventor: Yasuo MIYAJIMA, Utsunomiya-shi (JP)

(73) Assignees: KABUSHIKI KAISHA TOSHIBA, TOKYO (JP); TOSHIBA MEDICAL SYSTEMS CORPORATION, OTAWARA-SHI (JP)

(21) Appl. No.: 13/022,762

(22) Filed: Feb. 8, 2011

(30) Foreign Application Priority Data
Feb. 15, 2010 (JP) 2010-30688

(51) Int. Cl. A61B 8/00

(52) U.S. Cl. 600/459

(57) ABSTRACT

An ultrasound probe according to one embodiment includes a plurality of ultrasound vibrators, a switching part, and an amplification part. The ultrasound vibrator transmits ultrasound and receives ultrasound echoes reflected within a subject. The switching part is configured to cause aperture movement of ultrasound beams by selectively switching signals from two or more of the ultrasound vibrators. The amplification part is configured to amplify signals from the switching part. The ultrasound probe receives signals from the plurality of ultrasound vibrators in a time-division manner by controlling the switching part. In addition, the ultrasound probe further comprises an impedance transforming part. The impedance transforming part is configured to be interposed between the ultrasound vibrator and the switching part, receive signals from the ultrasound vibrator at high impedance, and output such to the switching part at low impedance.

### CROSS-REFERENCE TO RELATED APPLICATION

This application is based upon and claims the benefit of priority from Japanese Patent Application No. 2010-030688, filed Feb. 15, 2010; the entire contents of which are incorporated herein by reference.

### FIELD

Embodiments described herein relate generally to an ultrasound probe that is connected to an ultrasound imaging apparatus and transmits/receives ultrasound to/from a subject, and specifically a technology in which an amplifier and a delay circuit are shared among a plurality of ultrasound vibrators by switching among a plurality of ultrasound vibrators.

### BACKGROUND

In a 3D scannable ultrasound imaging apparatus, it is necessary to steer transceiving beams in two axial directions by using a 2D array transducer, or steer one axial direction and move an aperture in the other axial direction (hereinafter referred to as “aperture movement”).

In the 2D array transducer, because vibrators require a 2D array, the number of vibrators significantly increases and thus the number of vibrators required is one order of magnitude (1000 or more) more than the conventional number of vibrators (200 or less).

In addition, with 2D array vibrators, a method is known to form an image by, from the center of a beam, steering the beam in one direction, in order to provide a fan shaped image that is used for the heart, etc. This method of steering the direction of a beam is effective when forming an image of the subject from a small gap such as between ribs, although there is a problem that in cases of imaging the abdominal parts or superficial organs, it is difficult to detect a lesion in the vicinity of the body surface unless a wide field of view around the body surface is obtained.

Therefore, it is desirable to provide an image shaped like a rectangle, trapezoid, parallelogram, or fan face of a folding fan by forming an image while not only steering beams but also moving the aperture. With this aperture movement, in order to obtain a wider field of view on the surface of the body, it is desirable to increase the number of vibrators (36 to 128 in the method of steering the direction of beams, and 128 to 192 in the method of moving an aperture), and thus even if the channel of the main body is set at 64CH in a conventional 1D array probe, a probe having 128 or more vibrators is used.

In this manner, in an ultrasound 2D array probe, the more the number of vibrators increases, the more the number of connection cables to the probe and reception circuits of the apparatus significantly increases, where due to the practical size, weight, and cost, it would often be a problem to implement it. Therefore, with the aim of suppressing power consumption and reducing the circuit size, a configuration may be applied in which an amplifier circuit or a delay circuit associated with one channel is shared among a plurality of vibrators.

The configuration of a conventional ultrasound probe and ultrasound imaging apparatus in which an amplifier circuit or a delay circuit is shared among a plurality of vibrators is described with reference to FIG. 4 and FIG. 5. FIG. 4 is a functional block diagram showing a configuration with a focus on the reception part of a general ultrasound imaging apparatus. In addition, FIG. 5 is a diagram describing the configuration of a reception circuit part of a conventional ultrasound probe that moves the aperture of an ultrasound beam by switching among a plurality of ultrasound vibrators.

An ultrasound probe 1 is composed of a transmission circuit 10, a group of ultrasound vibrators 11, an amplifier circuit 12, a delay circuit 13, and an summation circuit 14.

Although the transmission circuit 10 is not shown, it is composed of a clock generator, a divider, a transmission delay circuit, and a pulsar. The clock pulse generated in the clock generator is devided to a rate pulse, for example, approximately 5 KHz by the divider. This rate pulse is provided through the transmission delay circuit to the pulsar to generate voltage pulse at high frequency and drive (vibrate mechanically) a group of ultrasound vibrators 11.

Thereby, in response to electrical signals from the transmission circuit 10, ultrasound beams are radiated from the group of ultrasound vibrators 11 towards a subject to be observed.

The group of ultrasound vibrators 11 is, for example, configured so as to be aligned in an N×M array, and transceives ultrasound from/to a subject to be observed (for example, the heart). Ultrasound beams transmitted from each of the ultrasound vibrators (hereinafter referred to as “each vibrator”) that make up the group of ultrasound vibrators 11 reflect in response to the structure and motions, etc. in the subject to be observed at an interface between different acoustic impedances such as the border of structures in the subject to be observed.

The amplifier circuit 12 executes processing such as low noise amplifying or buffering in order to detect well any imperceptible ultrasound echo signals received at each vibrator of the group of ultrasound vibrators 11. The configuration of the amplifier circuit 12 is now described with reference to FIG. 5. As shown in FIG. 5, the amplifier circuit 12 is composed of a preamplifier 121, a switching part 122, and a variable gain amplifier 123.

The switching part 122, in response to the control from a controlling part (not shown), switches and outputs, in accordance with aperture movement, signals to be input to the preamplifier 121 from each vibrator that makes up the group of ultrasound vibrators 11. In the case of an ultrasound probe that moves the aperture by switching among a plurality of ultrasound vibrators, with switching control per scan line of the switching part 122, signals from each vibrator are transmitted, via the delay circuit 13 and the summation circuit 14, to a reception part of the ultrasound imaging apparatus main body 2 by selecting elements per scanning line. Thereby, it is possible to share the preamplifier 121, the variable gain amplifier 123, and the delay circuit 13 among the plurality of vibrators (the preamplifier 121, the variable gain amplifier 123, the delay circuit 13, and the summation circuit 14 are described blow).

It should be noted that FIG. 5 shows the case of a 2D array vibrator having 32 lines and 64 rows of vibrators, in which a vibrator at the fifth line and the first row is indicated as E501, and E533 indicates a vibrator at the fifth line and the 33^{rd} row. In addition, for each switch that makes up the switching part 122, a switch that switches to vibrator E501 is indicated as S501, and a switch that corresponds to the vibrator E533 is indicated as S533.

The variable gain amplifier 123 has a function, so-called TGC (Time Gain Control), that varies amplification degrees temporally. The level of signals from each vibrator varies depending on distance from each vibrator to a subject, in which the further the distance is, the longer the time of reflected waves arrives, and the level of signals from the corresponding vibrator becomes lower because the reflected waves are attenuated. Therefore, the variable gain amplifier 123 can amplify temporally different level of signals to a constant level of signals so as to output by changing amplification degrees depending on the distance between an ultrasound vibrator and the subject.

The preamplifier 121 is a so-called LNA (Low Noise Amplifier), and typically, a low noise amplifier with fixed gain is used.

The circuit configuration of the preamplifier 121 is further described in detail with reference to FIG. 6. FIG. 6 is an example of a circuit diagram of parts of the group of ultrasound vibrators and the preamplifier of a conventional ultrasound probe that shares an amplifier and a delay circuit among a plurality of ultrasound vibrators.

A signal source V1 and a resistance Rs1 as well as a signal source V2 and a resistance Rs2 equivalently show each vibrator that makes up the group of ultrasound vibrators 11. For example, as described with reference to FIG. 5 and FIG. 6 as examples, the vibrator E501 in FIG. 5 corresponds to the signal source V1 and resistance Rs1 in FIG. 6 and the vibrator E533 corresponds to the signal source V2 and resistance Rs2.

In addition, the switch U3 and the switch U4 in FIG. 6 show each switch (switch S501 and S533) that makes up the switching part 122 in FIG. 5, in which for example, the switch S501 in FIG. 5 corresponds to the switch U3 and the switch S533 corresponds to the switch U4 in FIG. 6.

The preamplifier 121 is composed of transistors (FET: Field Effect Transistor) M1 and M3 in FIG. 6. The gate potential of the transistor M1 is supplied by a power supply V_{bias}. A power supply V_{dd} is connected to the drain and a current source I1 is connected to the source of the transistor M1 so as to make up a drain grounded amplifier circuit (source follower). The output of the transistor M1 is connected to the gate of the transistor M3. The transistor M3 makes up a source grounded circuit so as to amplify the output of the transistor M1. It should be noted that the gate of the transistor M3 has high impedance. Therefore, a capacitor C3 is equipped for the gate of the transistor M3, in which this capacitor C3 maintains bias and reduces noise in cases of leakage due to the switches U3 and U4 (leakage due to coupling capacitance of an OFF switch) or no vibrator being selected.

It should be noted that as shown in FIG. 6, a high-pass filter that is composed of the capacitor C1 and the resistance R4 (or the capacitor C2 and the resistance R6) may be equipped between each switch (switch U3 or switch U4) that makes up the switching part 122 and the transistor M1 so as to transit only higher harmonics. The capacitor C1 (or C2) has an effect of making high frequency waves transit easily, in which the lower limit frequency to transit through the high-pass filter is proportional to 1/C1R4 (or 1/C2R6).

As a technology for obtaining a stable image by transiting only higher harmonics with the equipped high-pass filter as mentioned above, even in cases in which for example, an image is difficult to generate due to the lack of uniformity of the body tissue involved in aging or increased lipid layers, THI (Tissue Hermonic Imaging) is known.

At this point, reference is made to FIG. 4. The signals amplified by the amplifier circuit 12 are provided a delay time by the delay circuit 13, summed by the summation circuit 14, and output to the reception part of the ultrasound imaging apparatus main body 2.

Thereby, it is possible to reduce the number of output signal lines from the ultrasound probe 1. In other words, the number of the signal lines within the probe cable is reduced.

The reception part of the ultrasound imaging apparatus main body 2 is composed of a reception main delay circuit 20, a signal processing part 21, an image processing part 22, and a display part 23.

The reception main delay circuit 20 is composed of a delay and summation circuit such as a digital beamformer unit so as to receive signals from the ultrasound probe 1 and phase and sum the signals. At this time, an amplifier circuit such as a preamplifier may be equipped on the input side of the delay and summation circuit, in which after the signals are amplified by the amplifier circuit, phasing and adding are performed.

The signals phased and summed by the reception main delay circuit 20 are detected by the signal processing part 21 so as to extract an envelope, and furthermore this extracted envelope is displayed on a display part 23 after being coordinate-transformed according to the cross-section of the subject to be observed and gradation-processed suitably for image displaying by the image processing part 22.

Thereby, morphological information within the subject to be observed is displayed on the display part 23 in real time.

In light of the configuration of the abovementioned ultrasound probe, the mechanism of the aperture movement by switching among a plurality of vibrators is described with reference to FIG. 5 by way of example of an ultrasound 2D array probe.

For example, given that the number of rows is set to be 32 rows that receive simultaneously per transmission, if the first row through the 32^{nd} row are used for receiving, the vibrators of the 33^{rd} row need not to be used, and if the second row through the 33^{rd} row are used for receiving, the vibrators of the first row need not be used. In other words, as shown in FIG. 5, the vibrators of the first row and the vibrators of the 33^{rd} row are not simultaneously used, and thus it is possible to share the variable gain amplifier 123 and the delay circuit 13, which consume lots of power, among the vibrators.

In cases of configurations in which the variable gain amplifier 123 or the delay circuit 13 is shared among a plurality of vibrators by the abovementioned switching control by the switching part 122, the preamplifier 121 requires high input impedance and excellent noise characteristics.

However, as shown in FIG. 5, in cases of configurations in which the switching part 122, so as to share the variable gain amplifier 123 and delay circuit 13 among a plurality of vibrators, is equipped on the input side of the preamplifier 121, because the vibrators in the ultrasound 2D array probe have higher electrical impedance than those of the vibrators in a conventional ultrasound probe, effects due to floating capacitance of each switch (S501 and S533) that makes up the switching part 122 (impedance drop) result in degradation of receiving performance.

This is because the vibrators of a conventional probe have impedance corresponding to the capacitance 50 to 60 pF, while in cases of the vibrators of a 2D array probe, because a number of vibrators needs to be disposed within a certain region, the area of an individual vibrator is small and the capacitance is as low as 5 pF, resulting in higher impedance than that of a conventional vibrator. Therefore, the floating capacitance of each switch that makes up the switching part 122 turns out to be higher than the capacitance of the vibrator, which is not insignificant.

Reference is now made to FIG. 7. FIG. 7 shows an example of the configuration of a reception part in a conventional ultrasound probe in which a variable gain amplifier 123 is equipped for each vibrator that makes up the group of ultrasound vibrators 11 and a delay circuit is shared among a plurality of vibrators.

The output impedance of the variable gain amplifier 123 is lower than that of each vibrator that makes up the group of ultrasound vibrators 11. Therefore, as shown in FIG. 7, it is possible to reduce degradation of the receiving performance due to the floating capacitance of each switch that makes up the switching part 122 by providing a variable gain amplifier 123 for each vibrator that makes up the group of ultrasound vibrators 11. However, the number of variable amplifiers 123 needs to correspond to the number of vibrators, resulting in a problem of insufficiently reduced power consumption and also increasing the size of the circuit.

Although several approaches have been contemplated, e.g. implementing a common connection circuit within an ultrasound probe so as to reduce the number of cables, the reception circuit implemented in the ultrasound probe was unavoidably subjected to constraints on receiving quality, such as degradation in noise characteristics due to the power consumption and the implemention area. Additionally, an approach in which the reception circuit of the ultrasound imaging apparatus main body is connected to a plurality of vibrators has been suggested, but it requires a number of switches to connect to the vibrators or it results in a problem of addition with incorrect delay, when connectable vibrators are scattered. Because each vibrator is a passive device and signals are output as noise from the circuit that makes up the vibrator even when not in use, scattered connectable vibrators cause the problems of increasing noise from circuits that make up the vibrators not in use, or of decreasing the sensitivity.

### BRIEF DESCRIPTION OF THE DRAWINGS

FIG. 1 is a diagram describing a configuration of a reception part of the ultrasound probe according to an embodiment.

FIG. 2 is a circuit diagram showing configurations of a group of ultrasound vibrators, an impedance transforming part, and a preamplifier of the ultrasound probe according to the embodiment.

FIG. 3 is a circuit diagram showing configurations of a group of ultrasound vibrators, an impedance transforming part, and a preamplifier of the ultrasound probe according to a variation.

FIG. 4 is a functional block diagram showing a configuration of a reception part of an ultrasound probe and an ultrasound imaging apparatus.

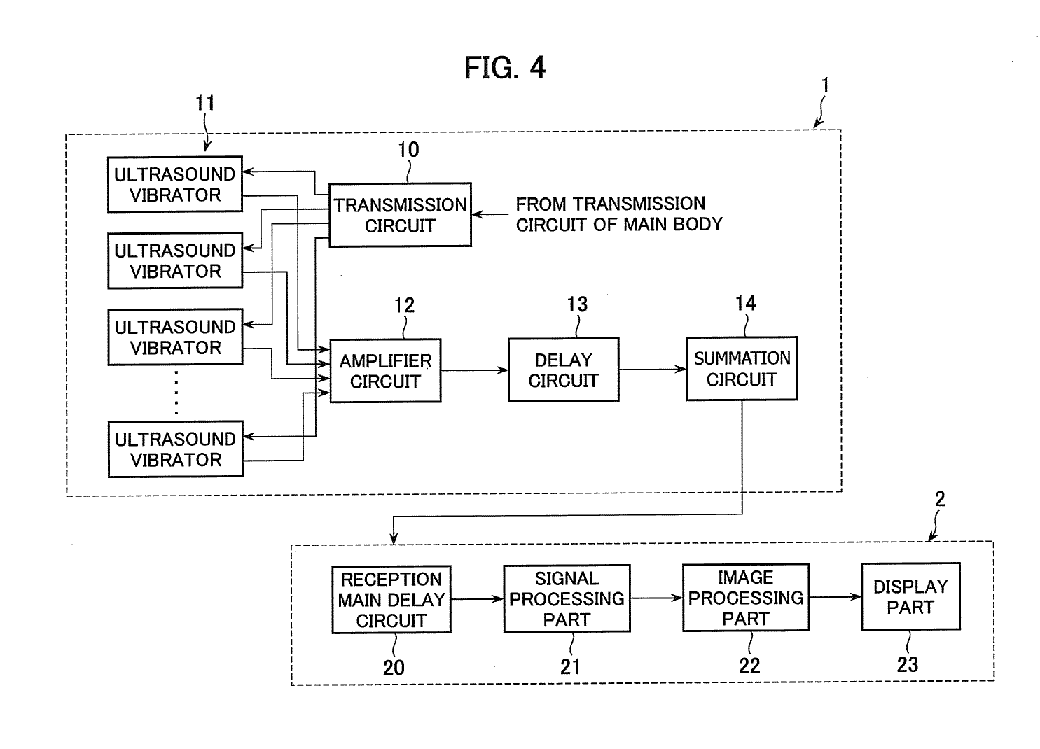

FIG. 5 is a diagram describing a configuration of a reception part of a conventional ultrasound probe in which an amplifier and a delay circuit are shared among a plurality of ultrasound vibrators.

FIG. 6 is an example of a circuit diagram of parts of a group of ultrasound vibrators and an amplifier circuit of a conventional ultrasound probe in which an amplifier and a delay circuit are shared among a plurality of ultrasound vibrators.

FIG. 7 is a diagram describing a configuration of a reception part of a conventional ultrasound probe in which an amplifier is equipped for each vibrator and a delay circuit is shared among a plurality of vibrators.

### DETAILED DESCRIPTION

An object of one embodiment is to provide an ultrasound probe that is capable of, while suppressing power consumption by an amplifier and significant increase in the size of circuits, preventing degradation of receiving performance due to floating capacitance of the switching part and obtaining excellent receiving quality through switching control by a switching part even in configurations in which an amplifier and a delay circuit are shared among a plurality of vibrators.

According to one embodiment, an ultrasound probe includes a plurality of ultrasound vibrators, a switching part, and an amplification part. The ultrasound vibrator is configured to transmit ultrasound and receive ultrasound echoes reflected within a subject. The switching part is configured to cause aperture movement of ultrasound beams by selectively switching signals from two or more of the ultrasound vibrators. The amplification part is configured to amplify signals from the switching part. The ultrasound probe receives signals from the plurality of ultrasound vibrators in a time-division manner by controlling the switching part. In addition, the ultrasound probe further comprises an impedance transforming part. The impedance transforming part is configured to be interposed between the ultrasound vibrator and the switching part, receive signals from the ultrasound vibrator at high impedance, and output such to the switching part at low impedance.

Various embodiments will be described hereinafter with reference to the accompanying drawings.

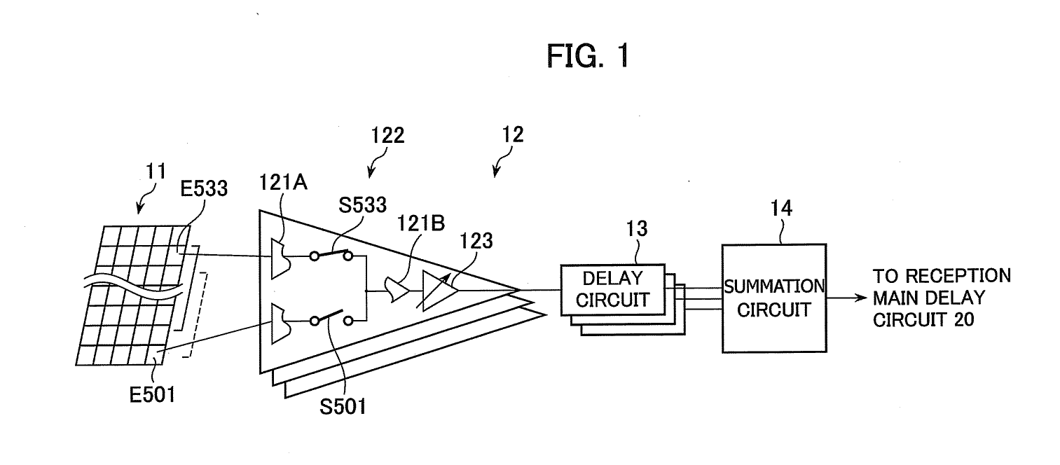

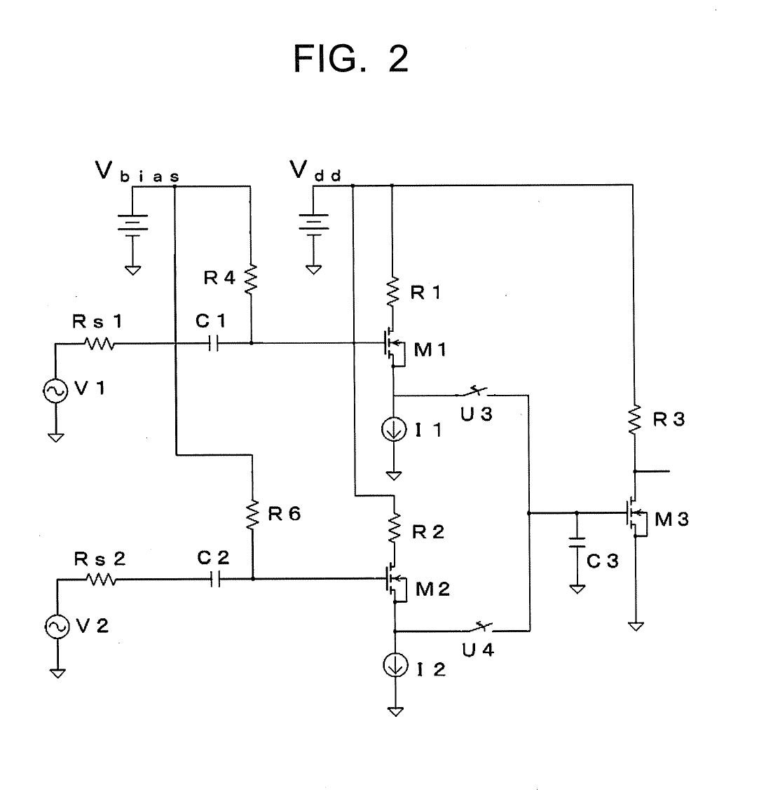

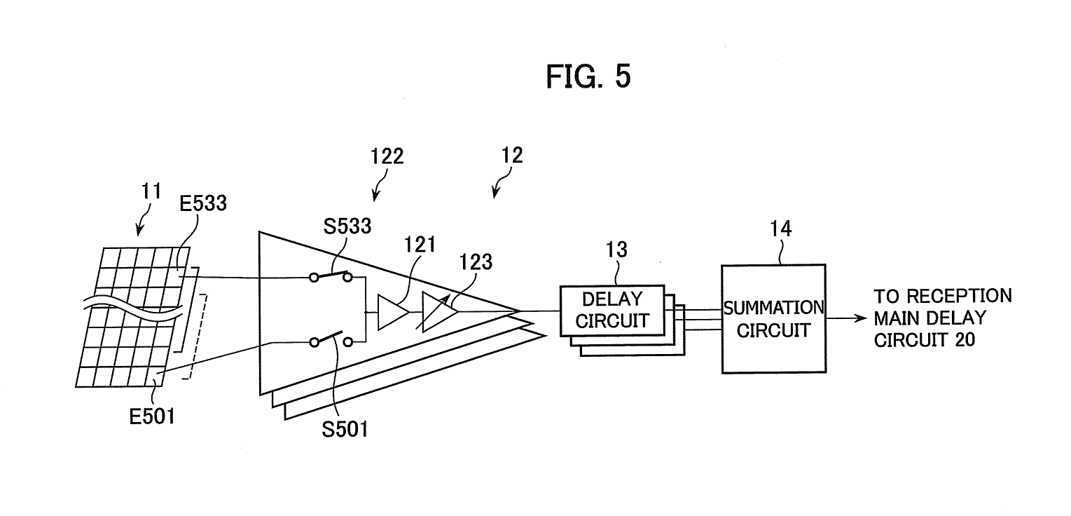

The configuration of the ultrasound probe according to an embodiment is described with reference to FIG. 1 and FIG. 2. FIG. 1 is a diagram describing the configuration of a reception part of the ultrasound probe according to the embodiment. FIG. 2 is a circuit diagram showing the configurations of a group of ultrasound vibrators, an impedance transforming part, and a preamplifier of the ultrasound probe according to the embodiment. It should be noted that in the description of the configuration according to the embodiment, different configurations of the amplifier circuit 12 from a conventional ultrasound probe are focused upon. The configurations of the group of ultrasound vibrators 11, the delay circuit 13, and the summation circuit 14 are similar to those of a conventional ultrasound probe, as shown in FIG. 5.

As shown in FIG. 1, the amplifier circuit 12 in the ultrasound probe according to the embodiment is composed of an impedance transforming part 121A, a switching part 122, a preamplifier 121B, and a variable gain amplifier 123.

In FIG. 2, each vibrator is shown by replacing it with an equivalent signal source (power supply) V and resistance Rs corresponding to output impedance. For example, as described with reference to FIG. 1 and FIG. 2 as examples, a vibrator E501 in FIG. 1 corresponds to a signal source V1 and a resistance Rs1 in FIG. 2 and a vibrator E533 corresponds to a signal source V2 and a resistance Rs2.

The signals from each vibrator (for example, vibrators E501 and E533) that makes up the group of ultrasound vibrators 11 are input to the impedance transforming part 121A that is provided for each vibrator. The impedance transforming part 121A is composed of a device with high input impedance and low output impedance.

The impedance transforming part 121A is specifically described with reference to FIG. 2. The impedance transforming part 121A corresponds to transistors (FET) M1 and M2 in FIG. 2. Each of the transistors M1 and M2 is connected to each vibrator that makes up the group of ultrasound vibrators 11. It should be noted that the description below is such that, as shown in FIG. 2, the impedance transforming part 121A corresponding to the transistor M1 is connected to the vibrator E501 shown with the signal source V1 and the resistance Rs1, and the impedance transforming part 121A corresponding to the transistor M2 is connected to the vibrator E533 shown with signal source V2 and resistance Rs2.

The gate potential of the transistors M1 and M2 is supplied by a power supply V_{bias}. A power supply V_{dd} is connected to the drain and a current source I1 is connected to the source of the transistor M1 so as to make up a drain grounded amplifier circuit (source follower).

Thereby, the impedance on the input side (high impedance) is transformed into low impedance on the output side of the transistors M1 and M2.

It should be noted that as shown in FIG. 2, for example, a high-pass filter that is composed of a capacitor C1 and a resistance R4 may be equipped between the vibrator E501 shown with the signal source V1 and the resistance Rs1 and the transistor M1 so as to transit only higher harmonics. At this time, the capacitor C1 has an effect of making high frequency waves transit easily, in which the frequency to transit through the high-pass filter is proportional to 1/C1R4. In the example of FIG. 2, with the configuration comprising the capacitor C1, the resistance R4, and the power supply V_{bias}, the functions of the high-pass filter and bias of gate potential in the transistor M1 are combined. Similarly, the high-pass filter that is composed of the capacitor C2 and the resistance R6 corresponds to the transistor M2.

The switching part 122 is equipped on the output side of the impedance transforming part 121A, in which the signals output from the impedance transforming part 121A (transistors M1 and M2) are input into the switching part 122.

The switching part 122, in response to the control by a controlling part (not shown), switches the signals output via the impedance transforming part 121A from each vibrator that makes up the group of ultrasound vibrators 11 in accordance with aperture movement, and outputs to the preamplifier 121B.

At this time, with switching control by the switching part 122, signals from each vibrator are transmitted to the reception part of the ultrasound imaging apparatus main body 2 via the delay circuit 13 and the summation circuit 14 in a time-division manner. Thereby, it is possible to share the preamplifier 121B, the variable gain amplifier 123, and the delay circuit 13 among a plurality of vibrators (the preamplifier 121B and the variable gain amplifier 123 are described below).

It should be noted that the switch U3 and the switch U4 in FIG. 2 show each switch (switch S501 and S533) that makes up the switching part 122 in FIG. 1. In the description below, the switch U3 connected to the transistor M1 corresponds to the switch S501 in FIG. 1 and the switch U4 connected to the transistor M2 corresponds to the switch S533 in FIG. 1.

The preamplifier 121B is a circuit to receive input from the switching part 122 and to amplify and output signals, and typically a low noise amplifier with fixed gain, known as an LNA, is used.

The preamplifier 121B is specifically described with reference to FIG. 2. The preamplifier 121B corresponds to the transistor M3 in FIG. 2. To the gate of the transistor M3, signals output from the transistor M1 or M2 are input in response to switching control by the switches U3 and U4 that make up the switching part 122. The transistor M3 makes up the source grounded circuit and amplifies the signals input. It should be noted that the gate of the transistor M3 has higher impedance. Therefore, a capacitor C3 is equipped for the gate of the transistor M3, in which this capacitor C3 reduces leakage due to the switches U3 and U4 (leakage due to coupling capacitance of an OFF switch), and maintains bias and reduces noise in cases of no vibrator being selected.

The signals amplified by the preamplifier 121B are input to the variable gain amplifier 123. The variable gain amplifier 123 is controlled by a controlling part (not shown) as a TGC (Time Gain Control) that is a temporal variation of amplification degree. Because the configuration of the variable gain amplifier 123 is similar to the conventional one shown in FIG. 5, a detailed description is omitted.

The signals output from the variable gain amplifier 123 are provided with delay time by the delay circuit 13, summed by the summation circuit 14, and output to the reception part of the ultrasound imaging apparatus main body 2.

Based on the abovementioned ultrasound probe according to the embodiment, the impedance transforming part 121A transforms the impedance on the input side up (high impedance) and the impedance on the output side of the impedance transforming part 121A into low impedance. Thereby, it is possible to use a vibrator with small area (with small capacitance and high impedance) in an ultrasound 2D array probe, etc., for each vibrator that makes up the group of ultrasound vibrators, while suppressing effects of floating capacitance due to switches (for example, S501 or S533) that make up the switching part 122 and obtaining excellent receiving quality.

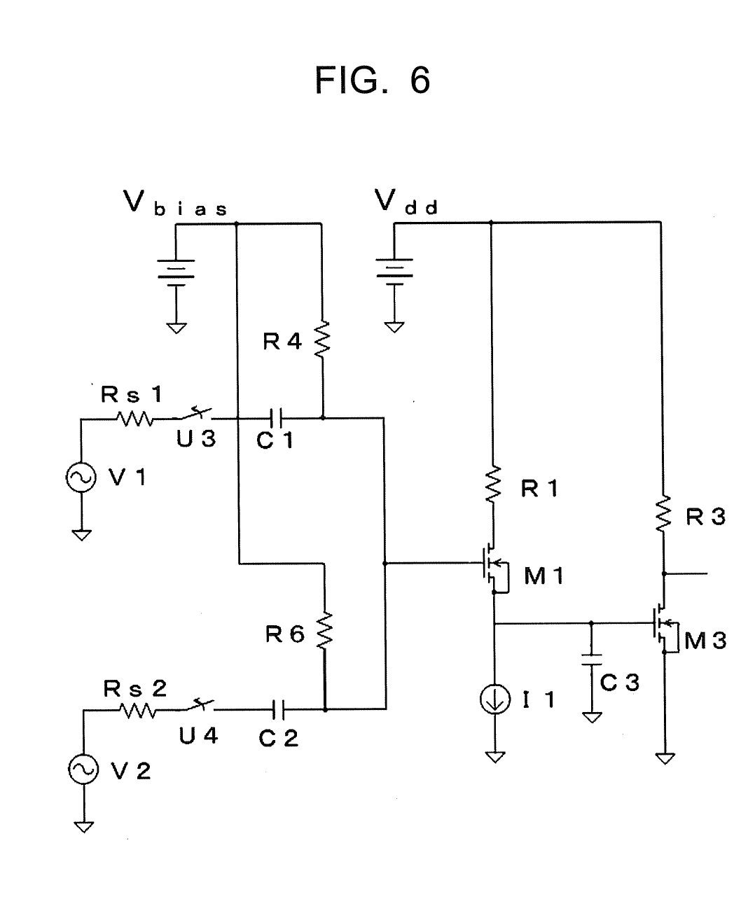

In addition, the transistors M1 and M2 that make up the impedance transforming part 121A have similar configuration of the transistor M1 that makes up the preamplifier 121 of a conventional ultrasound probe shown in FIG. 6. Therefore, it is possible to achieve the abovementioned enhancement of receiving quality only by increasing the power and circuit corresponding to the transistor M2 to a conventional ultrasound probe.

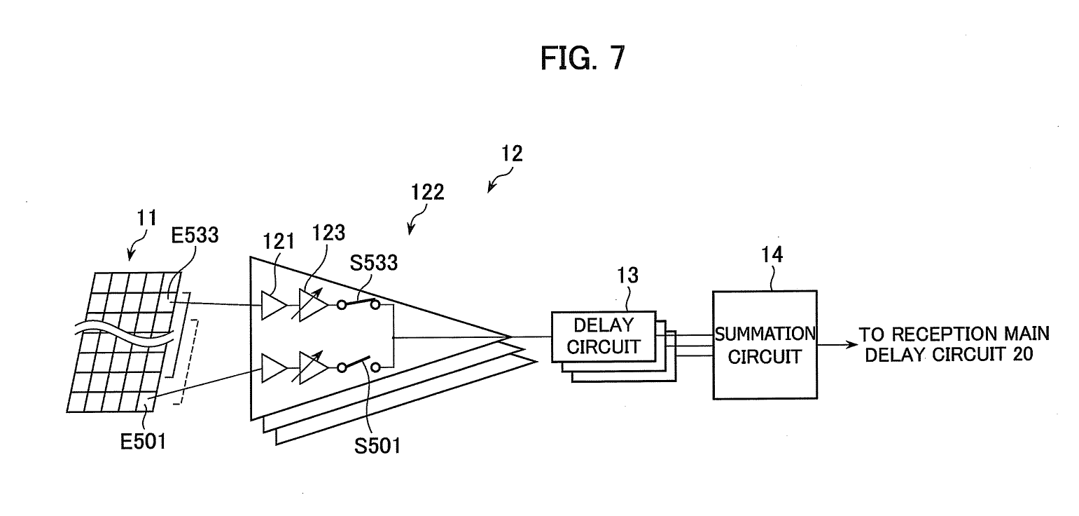

The transistors M1 and M2 that make up the impedance transforming part 121A can be operated at less power and have smaller circuit size than those of the variable gain amplifier 123. Therefore, as shown in FIG. 7, it is possible to obtain the effect of enhancing the abovementioned receiving quality with less power increase than that of the configuration in which the variable gain amplifier 123 is equipped per vibrator that makes up the group of ultrasound vibrators 11 and to suppress the increase in the size of the circuit to a small extent.

It should be noted that as the preamplifier 121B, the preamplifier 121 (composed of transistors M1 and M3) in a conventional ultrasound probe, as shown in FIG. 5 and FIG. 6, may be used. In addition, because the abovementioned impedance transforming part 121A, switching part 122, variable gain amplifier 123, delay circuit 13, and summation circuit 14 are implemented within the limited space in the ultrasound probe, it is desirable to implement them as an integrated circuit, including a control circuit in which those circuits operate.

In addition, while an example in which MOSFET (Metal-Oxide-Semiconductor Field-Effect Transistor) is used as transistors M1, M2, and M3 that make up the impedance transforming part 121A and the preamplifier 121B was described above, if it is a device that can provide a similar effect, the configuration is not limited to this, and for example, a bipolar transistor may be used.

In addition, while an example in which a source follower is used as the impedance transforming part 121A was described, if it is a circuit configuration with high input impedance and low output impedance, the configuration is not limited to this.

### (Variation)

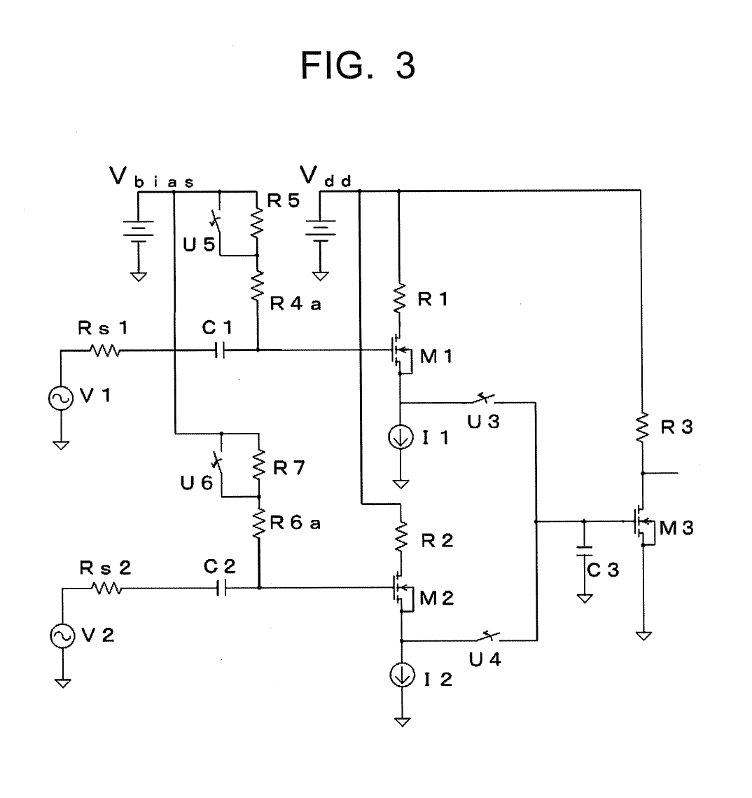

The configuration of the ultrasound probe according to a variation is described with reference to FIG. 3. FIG. 3 is a circuit diagram of parts of a group of ultrasound vibrators and a preamplifier of the ultrasound probe according to the variation.

The impedance transforming part 121A in the variation is different in that the part corresponding to the resistance R4 that makes up a high-pass filter of the impedance transforming part 121A according to the embodiment shown in FIG. 2 is composed of a resistance R4a, a resistance R5, and a switch U5 in FIG. 3 (similarly, the part corresponding to the resistance R6 in FIG. 2 is composed of a resistance R6a, a resistance R7, and a switch U6 in FIG. 3). In this description, configurations and operations of the high-pass filter of the impedance transforming part 121A that are different from those of the embodiment are focused.

In the impedance transforming part 121A according to the variation, the high-pass filter equipped on the input side of the transistor M1 is composed of a capacitor C1 and resistances R4a and R5. The capacitor C1 is similar to the capacitor C1 in the embodiment shown in FIG. 2.

In the impedance transforming part 121A according to the variation, the resistance R4 in the embodiment shown in FIG. 2 is composed by dividing it into the resistances R4a and R5 (i.e., the resistance value shows the relationship of R4=R4a+R5), and the switch U5 is equipped at the locations at which the resistance R5 is bypassed.

In that way, it is possible to change the cutoff frequency of the high-pass filter that is composed of the capacitor C1 and the resistances R4a and R5, by switching the switch U5.

Specifically, when the switch U5 is ON, the resistance R5 is short and the cutoff frequency of the high-pass filter becomes 1/(2π×R4a×C1), resulting in a value higher than the cutoff frequency when the switch U5 is OFF (1/{2π×(R4a+R5)×C1}).

The switching of the switch U5 operates in conjunction with the switching of the switch U3. When the switch U3 is ON (when the corresponding vibrator E501 is used), the switch U5 is switched to OFF so as to set the cutoff frequency lower. The capacitance of the capacitor C1 and the resistance value of the resistance R4a+R5 are adjusted so that the cutoff frequency 1/{2π×(R4a+R5)×C1} at this time becomes a frequency that can transit the band of the signals from the vibrator E501 in response to the reflected waves from a subject.

In addition, when the switch U3 is OFF (when the corresponding vibrator E501 is not used and the vibrator E533 corresponding to the switch U4 is used), the switch U5 is switched to ON so as to set the cutoff frequency higher. The capacitance of the capacitor C1 and the resistance value of the resistance R4a are adjusted so that the cutoff frequency 1/(2π×R4a×C1) at this time becomes a frequency that can block the band including signals from the vibrator E501 in response to the reflected waves from the subject.

It should be noted that switching of the switch U5, strictly, may occur when the corresponding vibrator E501 is selected, and is controlled so that switching of the switch U5 from ON to OFF is completed by the time the corresponding vibrator E501 transmits ultrasound towards a subject and starts receiving the reflected waves (reflected waves are thereby transmitted), and switching from OFF to ON is executed after completion of receiving the reflected waves.

In this manner, it is possible to reduce the amplitude of the signals (specifically, signals from a vibrator not in use, in other words, noise) supplied to the gate of the transistor M1 by turning the switch U5 ON, shorting the resistance R5, and increasing the cutoff frequency of the high-pass filter to a value higher than the band of the signals from the vibrator E501 when the corresponding vibrator (for example, vibrator E501) is not in use.

It should be noted that while a circuit that is connected to the vibrator E501 shown with the signal source V1 and the resistance Rs1 was described above as an example, the circuit that is connected to the vibrator E533 shown with the signal source V2 and the resistance Rs2 is similar and corresponds to a capacitor C2, a resistance R6a, a resistance R7, and a switch U6.

According to the ultrasound probe of the variation, the cutoff frequency of the high-pass filter that makes up the impedance transforming part 121A is configured so that its value changes by switching the switch U5 (or U6) in conjunction with the switching of the switch U3 (or U4). In this way, it is possible to control the cutoff frequency of the high-pass filter so that it is higher when the corresponding vibrator E501 (or E533) is not in use, and to further reduce leakage of the signals (noise) from the circuit corresponding to the vibrator E501 not in use.

Now suppose that, for example, it is necessary to suppress −50 dB of signals against leakage from the circuit corresponding to the vibrators not in use. At this time, in the ultrasound probe according to the embodiment, it is necessary to perform the suppression using the switch U3 (or U4) that makes up the switching part 122 and this may sometimes require a switch with less leakage.

In contrast, in the ultrasound probe according to the variation, if it is configured to be able to suppress −20 dB of signals by the high-pass filter of the impedance transforming part 121A, then it is possible to lower the suppression rate of signals by the switch U3 (or U4) to −30 dB. Therefore, as the abovementioned ultrasound probe according to the embodiment, it is not necessary to use a switch with less leakage and it is possible to inexpensively realize switching the ultrasound vibrators in response to aperture movement.

While certain embodiments have been described, these embodiments have been presented by way of example only, and are not intended to limit the scope of the inventions. Indeed, the novel systems described herein may be embodied in a variety of their forms; furthermore, various omissions, substitutions and changes in the form of the systems described herein may be made without departing from the spirit of the inventions. The accompanying claims and their equivalents are intended to cover such forms or modifications as would fall within the scope and spirit of the inventions.

What is claimed is:

1. An ultrasound probe, comprising:
a plurality of ultrasound vibrators configured to transmit ultrasound and receive ultrasound echoes reflected within a subject;
a switching part configured to cause aperture movement of ultrasound beams by selectively switching signals from two or more of said ultrasound vibrators; and
an amplification part configured to amplify signals from said switching part,
wherein the ultrasound probe receives signals from the plurality of ultrasound vibrators in a time-division manner by controlling said switching part, and
wherein the ultrasound probe further comprises an impedance transforming part configured to be interposed between said ultrasound vibrator and said switching part, receive signals from said ultrasound vibrator at high impedance, and output such to said switching part at low impedance.

2. The ultrasound probe according to claim 1, wherein said impedance transforming part further comprises a high-pass filter configured to remove signals at a predefined frequency from among the signals from said ultrasound vibrator.

3. The ultrasound probe according to claim 2, wherein said high-pass filter is configured to be able to change said predefined frequency so that it transits signals from said ultrasound vibrator that is selected by said switching part within the band, including the frequency that drives said ultrasound vibrator, and removes signals from said ultrasound vibrator that is not selected by said switching part within the band, including the frequency that drives said ultrasound vibrator.

4. The ultrasound probe according to claim 3, wherein said high-pass filter is composed of capacitance that forms AC coupling and resistance that also acts as resistance to provide bias, and the frequency at which transit is possible is switched by switching the value of the resistance to provide said bias.

5. The ultrasound probe according to claim 1, wherein said impedance transforming part is configured with a source follower.
